(19) United States
(12) Patent Application Publication
(10) Pub. No.: US 2004/0067534 A1
(43) Pub. Date: Apr. 8, 2004

(54) Methods of preparing improved agents by coevolution

(76) Inventor: Simon Delagrave, Avondale, PA (US)

Correspondence Address: Simon Delagrave, 709 Spencer Rd., Avondale, PA, 19311, US

(21) Appl. No.: 10/659,083

(22) Filed: Sep. 9, 2003

Related U.S. Application Data
(60) Provisional application No. 60/416,819, filed on Oct. 8, 2002.

(51) Int. Cl. G01N 33/53; G01N 33/543

(52) U.S. Cl. 435/7.1; 436/518

(57) ABSTRACT

The present invention provides methods of coevolving components of biological and other systems to prepare evolved components with improved or other desired properties. Coevolution processes can include one or more cycles of in vitro diversification and selection. In some embodiments, methods of coevolving involve antibodies or antigens for the preparation of therapeutics or vaccines.

### CROSS-REFERENCE TO RELATED APPLICATIONS

This application claims the priority benefit of U.S. Provisional App. No. 60/416,819, filed Oct. 8, 2002, which is incorporated herein by reference in its entirety.

### FIELD OF THE INVENTION

The present invention relates to, inter alia, methods of coevolving components of biological systems and the preparation of agents with improved or novel properties.

### BACKGROUND OF THE INVENTION

Pathogens such as the viruses causing AIDS and hepatitis C have the ability to evolve resistance to their hosts' defenses during an infection. In the last century, those defenses have come to include small molecule drugs and, more recently, biopharmaceuticals such as monoclonal antibodies. Humans and their pathogens are now embroiled in an evolutionary race wherein old antimicrobials become ineffective, and new drugs need to be discovered to replace them.

In the last decade, many methods have been developed to simulate in vitro, at an accelerated pace, the evolution of molecules. For example, it has been reported that macromolecules such as nucleic acids and polypeptides can be evolved in the laboratory according to arbitrary selection criteria (Cunningham, et al., Prot. Eng., 1987, 1, 319; Joyce, et al., Gene, 1989, 82, 83; Chen, et al., Biotechnology, 1991, 9, 1073; Barbas et al., Proc. Natl. Acad. Sci. USA, 1994, 91, 3809.) In vitro evolution approaches are increasingly being used to study drug resistance and experimental work has been carried out to better understand natural or semi-natural coevolution (see.e.g., Cowen, FEMS Microbiol. Lett., 2001, 204, 1; Orencia, et al., Nat. Struct. Biol., 2001, 8, 238; Yusa, et al., J. Virol., 2002, 76, 3031; Dawkins, et al., Proc. R. Soc. Lond. B Biol. Sci., 1979, 205, 489; Van Valen, Evol. Theory, 1973, 1, 1; Lenski, et al., Amer. Nat., 1985, 125, 585; Domingo, et al., Faseb J. 1996, 10, 589; and Wlotzka, et al., Chem. Biol., 1997, 4, 25.) In silico (computational) coevolution has been used to identify design strategies providing HIV protease inhibitors effective against a wide range of protease inhibitor-resistant mutants (see, e.g., Rosin, et al., Artif. Life, 1998, 4, 41; Rosin, et al., Proc. Natl. Acad. Sci. USA, 1999, 95, 1369; Rosin et al., J. Mol. Biol., 1999, 287, 77). Rosin and colleagues modeled the coevolution of HIV protease and peptide inhibitors by evaluating, via molecular modeling, the strength of the interaction between numerous pairs of inhibitors and inhibitor targets (protease variants). There are, however, intrinsic limitations to molecular modeling: only relatively simple calculations can be used to analyze the large number of interactions considered in this work. Moreover, like most peptides, the peptides considered in this study are unlikely to be used as therapeutics. As a result, the model's predictive power is limited because realism is sacrificed in exchange for computational efficiency.

Also reported are methods of creating an effective vaccine against the highly variable hepatitis C virus (HCV) (Puntoriero, et al., Embo. J., 1998, 17, 3521). The authors report the isolation of peptide antigens (mimotopes) potentially capable of eliciting a protective immune response against a broad cross-section of known HCV genotypes. In this study, a synthetically generated diversity of viral antigen sequences is selected, via phage display, against a diverse panel of natural antibodies (human HCV-positive sera) representing several HCV genotypes. The synthetic viral sequences binding to the greatest proportion of human sera were injected in mice and shown to generate antibodies cross-reactive against a wide variety of natural HCV variants. In this case, the ensemble of viral sequences was synthetic but derived from an extensive database of naturally occurring sequences. While this approach is well suited to the problem of pre-existing genetic diversity, it is not expected to address the issue of diversity induced in a virus population by the long-term use of an antiviral drug.

In EP 667917, Henco et al. report a strategy termed “evolutive vaccination” in which a pathogen is prepared by sequentially exposing it to antisera. It is well known that such a method does not lend itself to the preparation of a therapeutic or vaccine intended for use in humans unless antisera are prepared using human volunteers—a risky and costly proposition.

As is evidenced by the literature, there is a current need for new methods that can help counter drug resistance. For example, methods that can aid in the design of improved drugs such as, for example, antibacterials and antivirals, as well as help prepare new and more effective vaccines are in constant demand. Accordingly, the methods described herein help fulfill these and other needs.

### SUMMARY OF THE INVENTION

The present invention provides, in part, a method of countering the development of resistance in a parent target to a parent neutralizing agent or countering the development of neutralizing activity in a parent neutralizing agent to a parent target comprising coevolving the parent target and the parent neutralizing agent, wherein the coevolving comprises diversifying each of the parent target and the parent neutralizing agent in vitro.

The present invention further provides a method of preparing a neutralizing agent having a desired neutralization profile or a target having a desired resistance profile comprising:

a) coevolving a parent target and a parent neutralizing agent pair to generate a collection of evolved neutralizing agents and a collection of evolved targets;

b) cross testing members of the collections for neutralizing activity or resistance; and

c) identifying at least one member of the collection of evolved neutralizing agents having the desired neutralizing profile or identifying at least one member of the collection of evolved targets having the desired resistance profile.

The present invention further provides a method of coevolving, comprising:

a) diversifying in vitro a parent target and selecting at least one next generation target from a diversified population of targets resulting from the diversifying, wherein the selected at least one target has new or improved resistance to a parent neutralizing agent having neutralizing activity against the parent target; and

b) diversifying in vitro the parent neutralizing agent and selecting at least one next generation neutralizing agent from a diversified population of neutralizing agents resulting from the diversifying, wherein the selected at least one neutralizing agent has new or improved neutralizing activity against the selected at least one target.

The present invention further provides a method of coevolving, comprising:

a) diversifying a parent neutralizing agent and selecting at least one next generation neutralizing agent from a diversified population of neutralizing agents resulting from the diversifying, wherein the selected at least one neutralizing agent has new or improved neutralizing activity against a parent target having resistance to the parent neutralizing agent; and

b) diversifying the parent target and selecting at least one next generation target from a diversified population of targets resulting from the diversifying, wherein the selected at least one target has new or improved resistance to the selected at least one neutralizing agent.

The present invention further provides a method of coevolving a parent target and parent neutralizing agent to produce a collection of evolved targets and a collection of evolved neutralizing agents, comprising:

i) contacting the parent neutralizing agent with members of an initial population of targets generated from the parent target;

ii) selecting one or more resistant targets within the initial population, wherein the one or more resistant targets has new or improved resistance to the parent neutralizing agent;

iii) diversifying the parent neutralizing agent to create a population of neutralizing agents;

iv) contacting members of the population of neutralizing agents with the one or more resistant targets selected in step ii);

v) selecting one or more further neutralizing agents from the population of neutralizing agents, wherein the one or more further neutralizing agents have new or improved neutralizing activity against the one or more resistant targets;

vi) optionally repeating steps i) and ii) or optionally repeating steps i), ii), iii), iv), and v) using the one or more further neutralizing agents in place of the parent neutralizing agent and using the one or more resistant targets in place of the parent target,

wherein the parent neutralizing agent and the further neutralizing agents are members of a collection of evolved neutralizing agents, and wherein the parent target and the resistant targets are members of a collection of evolved targets.

The present invention further provides a method of developing a desired characteristic in a parent antibody or parent target comprising coevolving the parent antibody and parent target, wherein the coevolving comprises diversifying each of the parent antibody or parent target in vitro.

The present invention further provides a method of preparing an antibody having broadened neutralizing activity compared with a parent antibody comprising coevolving the parent antibody and a parent target.

The present invention further provides a method of preparing an antigen having broadened antigenic activity compared with a parent antigen comprising coevolving the parent antigen and a parent neutralizing agent.

### BRIEF DESCRIPTION OF THE DRAWINGS

FIG. 1 shows an example two-step coevolution scheme.

FIG. 2 shows an example outline of a coevolution scheme.

FIG. 3 shows multiple coevolution cycles (iterations).

FIG. 4 shows the characterization of coevolved drug specificity.

### DESCRIPTION OF EMBODIMENTS OF THE INVENTION

The present invention provides, inter alia, methods for developing desired characteristics in one or more components of a multi-component system using coevolution techniques that involve the in vitro diversification of two or more components of the system. Systems can include, for example, systems of two, three, four, or more components, where each component can interact with at least one other component of the system. In some embodiments, the system is binary, comprising two components that interact with each other, forming an interactive pair.

Multi-component systems of the present invention can be biological systems, such as, for example, systems containing at least one component that is an organism or derived from an organism. Components can include, for example, cells, proteins, nucleic acids, small molecules, viruses, multicellular organisms, and the like. Some components can also be of non-biological origin such as, for example, synthetic molecules that can be used as drugs.

Coevolution of the components of a multi-component system can improve or change characteristics of one or more components by employing iterative diversification and selection cycles, where the results of earlier diversification and selection cycles can influence the outcome of subsequent diversification and selection cycles. According to some embodiments, each diversification and selection cycle can generate an improvement in a desired characteristic for a system component over a previous result from an earlier cycle, or over a baseline (starting) characteristic. In embodiments involving a binary system (two components), coevolution can include diversification and selection cycles that alternate between the two components. For example, a member having an improved characteristic that is identified after a diversification and selection cycle can be used to generate and improve characteristics connected with the other component in a subsequent diversification and selection cycle.

In some embodiments, the present invention provides methods for preparing improved components of interactive pairs (e.g., a two component system) using coevolution techniques. Interactive pairs can be, for example, any pair of system components that interact with each other and/or where at least one member of the pair modulates activity of the other member. Some examples of interactive pairs include receptor/ligand, target/neutralizing agent, pathogen/neutralizing agent, antigen/antibody, substrate/enzyme, ligand/ligand-binding protein, drug/drug-binding molecule, nucleic acid/nucleic acid-binding protein, nucleic acid/nucleic acid-binding drug, nucleic acid/nucleic acid (e.g., single-stranded nucleic acid/complementary nucleic acid), and the like. Some further examples of interactive pairs include a phosphorylated segment of a protein and an SH3 domain as well as a C-terminus of a polypeptide and a PDZ domain.

Certain characteristics of components of the interactive pair can be changed or improved upon using coevolution. For example, the binding properties of a ligand to a receptor can be changed, e.g., by increasing or decreasing ligand binding affinity or changing ligand binding specificity. In another example, neutralizing activity of an antibody towards a pathogen can be modulated, e.g., by increasing/decreasing antibody binding affinity or changing antibody specificity. In some embodiments, specificity of an interaction can be changed in a desired direction. For example, if a characteristic of a drug target changes, a drug can be changed so that it recognizes the changed target. Further, coevolution can create ligand/receptor pairs that have low or no cross-talk with other ligand/receptor pairs in an organism or cell, thereby enabling the formation of new signal transduction pathways. Additionally, coevolution can modify a drug so that it can neutralize a pathogen having developed a resistance to the drug.

In some embodiments, such as when interactive pairs include target/neutralizing agent pairs, the present invention provides methods for countering the development of resistance in a target to a neutralizing agent, or countering the development of neutralizing activity in a neutralizing agent to a target, using coevolution techniques. The methods can be used, for example, to prepare neutralizing agents with superior neutralizing activity that would be useful, for example, in treating diseases such as those that have become resistant to traditional treatment regimens.

The methods of the present invention can also be used in diagnostics. For example, coevolution can be used to prepare reagents that are better able to detect variants that can arise in a population due to exposure to a drug. Coevolution methods can also be used as part of quality assurance (QA)/quality control (QC) of, for example, live viral vaccines. To illustrate, the live viral vaccines can be grown in the presence of neutralizing antibodies against the live viral vaccine to detect the possible presence of adventitious agents (e.g., a virus other than the live viral vaccine). If cytopathic effects are observed while the live viral vaccine is grown in the presence of a neutralizing antibody, these effects may indicate the presence of adventitious agents. However, if resistance to the neutralizing antibodies occurs during testing for adventitious agents, cytopathic effects may be observed due to the growth of live viral vaccine escape mutants. Coevolved antibodies can be used, as neutralizing antibodies that are not susceptible to escape mutant selection, to prevent this phenomenom, thereby facilitating the QA/QC process.

Definitions

As used herein, “neutralizing agent” refers to any entity capable of wholly or partially neutralizing at least one activity of a target. A neutralizing agent can be a cell, protein, nucleic acid, small molecule, virus, multicellular organism, or the like. In some embodiments, the neutralizing agent can bind to a target. In some embodiments, a neutralizing agent is an antibody or related molecule.

As used herein, “neutralizing activity” refers to the ability of a neutralizing agent to counter or deactivate at least one activity of a target. For example, neutralizing activity can be an ability to antagonize a target such as by lessening the target's ability to perform at least one of its functions. Small molecules can neutralize a receptor protein by blocking binding with its ligand, or for example, an antibody can neutralize a pathogen by binding to an epitope displayed on the pathogen. Neutralizing activity can be whole or partial, that is, it can completely neutralize a target or merely reduce the target's activity.

Methods for measuring neutralizing activity are well known in the art and vary depending on the neutralizing agent/target system. For example, neutralizing activity of antibodies can be measured by a plaque reduction neutralization test (PRNT), or a microneutralization assay (see, e.g., Delagrave et al., Protein Engineering, 1999, 12, 357, which is incorporated herein by reference in its entirety). Neutralizing activity of other components, such as small molecules, polypeptides, and polynucleotides, can be measured, in the same way. Other methods for measuring neutralizing activity, such as in relation to a viral component, can include measuring inhibition of syncytium formation (RSV and HIV) or inhibition of cytopathic effects in virus-infected tissue culture.

As used herein, a “target” can be any entity having an activity or effect that can be neutralized wholly or partially by a neutralizing agent. A target can be a cell, protein, nucleic acid, small molecule, virus, multicellular organisms, or the like. In some embodiments, a target can be associated with a disease or disorder in a plant or animal (e.g., a mammal). For example, a target can be a nucleic acid or protein that is differentially expressed in certain cancer cells. In a further example, a target can be a disease-causing organism such as a virus, bacterium, fungus, or single-celled organism. In some embodiments, the target is an antigen. Targets can be characterized by the ability to develop resistance to a neutralizing agent.

As used herein, “resistance” refers to the ability of target to thwart or withstand the neutralizing activity of a neutralizing agent. Resistance is typically a characteristic that is developed in or acquired by in a target initially lacking or showing weak resistance. For example, a pathogen such as a virus or bacterium can develop resistance to a drug to which it was previously susceptible.

Resistance can be measured using, for example, neutralization assays such as are known in the art. If a pathogen fails to be neutralized by an agent at a concentration at which it is normally effective, then the pathogen can be considered resistant. For example, if it takes more antibody to neutralize a virus than normally observed, the virus can be considered to be resistant to the antibody. The increase in antibody can be about 2×, 3×, 4×, 5×, 10× or more than what is normally effective.

As used herein, “antigen” refers to an entity that can stimulate an immune response in an animal. An antigen can be a cell, protein, nucleic acid, small molecule, virus, multicellular organism, or the like. In some embodiments, the antigen is a protein, peptide, or epitope. In further embodiments, the antigen is a “preventative antigen.” A preventative antigen can prevent a disease or disorder with which the antigen is associated and can act, for example, as a vaccine.

As used herein, “antigenic activity” refers to the ability of an antigen to stimulate an immune response. Antigenic activity can be determined by measuring the immune system's response to the antigen using any appropriate assay known in the art. For example, one can immunize (inject antigen into) an animal and determine the antibody titer several days post-immunization. Antibody titers can be measured, for instance, by carrying out an ELISA against antigen coated on a multiwell plate.

As used herein, the term “parent” describes a component, such as a target or neutralizing agent, which is a starting component of a coevolution process. “Parent” distinguishes the starting components from evolved forms of the components derived from the coevolution process.

As used herein, “cell” refers to the constituent of tissues or cell lines, as well as single-celled organisms including bacteria, yeast, and fungi. Suitable cells according to the methods of the present invention can include, for example, immune cells (e.g., phagocytes), cancer cells, pathogen-infected cells, drug-producing cells (e.g., bacteria such as Streptomyces including S. fradiae which produces the polyketide antibiotic tylosin), engineered microorganisms (e.g., engineered E. coli), stem cells, fungi, bacteria, and the like. Some example bacteria include pathogenic bacteria such as Bacillus anthracis, Escherichia coli 0:157, Yersinia pestis, Helicobacter pylori, Clostridium difficile, Streptococcus pneumoniae, Staphylococcus aureus, Mycobacterium tuberculosis. Other bacteria can include bacterial agricultural pests. In some embodiments, bacteria can include Mycobacterium bovis vaccine strain (used as Mycobacterium tubeculosis vaccine as well as treatment for bladder cancer). In further embodiments, the cell can be other microbial pathogens such as Plasmodium falciparum (malaria) and the fungus Candida albicans.

As used herein, “polypeptides” or “proteins” are polymers of amino acids having, for example, from 2 to about 1000 or more amino acid residues. Any natureeally occurring or synthetic amino acid can form the polypeptide. Polypeptides can also include modifications such as glycosylations and other moieties. Example polypeptides include viral coat proteins, viral proteases, viral DNA or RNA polymerases, antibodies and fragments thereof, receptors, ligands, enzymes, toxins, prions, cytokines, hormones, fusion proteins in which a ligand-binding activity is fused to an effector function such as a toxin, antibodies covalently labeled with toxins or radioactive compounds or imaging reagents, enzymes which produce compounds having neutralizing activity, and the like. In some embodiments, polypeptides include specific cell proteins such as enzymes involved in drug production, cytokines in immune system cells, and proteins associated with viral infection. Proteins involved in attacking or recognizing pathogens (e.g., MHC molecules) can also be suitable.

As used herein, the term “antibody” includes polyclonal antibodies and monoclonal antibodies as well as fragments thereof. Antibodies include, but are not limited to mouse, rat, and rabbit, human, chimeric antibodies and the like. The term “antibody” also includes antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski, et al. Proc. Natl. Acad. Sci., 1985, 82, 8653 or Spira, et al., J. Immunol. Methods, 1984, 74, 307.

The invention also provides biologically active fragments of the polyclonal and monoclonal antibodies described above. These “antibody fragments” typically retain some ability to selectively bind with its antigen or immunogen. Such antibody fragments can include, but are not limited to: Fab, Fab′, F(ab′)₂, Fv, and SCA. An example of a biologically active antibody fragment is a CDR region of the antibody. Methods of making these fragments are known in the art, see for example, Harlow and Lane (1988), infra.

The antibodies of this invention also can be modified to create chimeric antibodies and humanized antibodies (Oi, et al., BioTechniques, 1986, 4(3), 214 which is incorporated herein by reference in its entirety). Chimeric antibodies are, for example, those in which the various domains of the antibodies' heavy and light chains are coded for by DNA from more than one species.

The isolation of other hybridomas secreting monoclonal antibodies with the specificity of the monoclonal antibodies of the invention can also be accomplished by one of ordinary skill in the art by producing anti-idiotypic antibodies (Herlyn, et al., Science, 1986, 232:100, which is incorporated herein by reference in its entirety). An anti-idiotypic antibody is an antibody which recognizes unique determinants present on the monoclonal antibody produced by the hybridoma of interest.

Antibodies according to the present invention can also include genetically engineered antibody fragments. For example, molecular clones of variable domains of antibodies can be transformed into single-chain variable domains (scFv), diabodies, Fab (Barbas et al., Proc. Natl. Acad. Sci. USA, 1992, 9, 10164), bivalent Fab (Fab′), etc., using standard recombinant DNA technology. Phage display (Smith, Science, 1985, 228, 1315), ribosome display (Hanes & Pluckthun, Proc. Natl. Acad. Sci. USA, 1997, 94, 4937) and mRNA display (Xu et al, Chem. Biol, 2002, 9, 933) can be used in vitro to select antibodies with desired affinity and/or specificity.

Laboratory methods for producing polyclonal antibodies and monoclonal antibodies, as well as deducing their corresponding nucleic acid sequences, are known in the art, see, e.g., ANTIBODIES, A LABORATORY MANUAL (Harlow and Lane eds. (1988)) and Sambrook et al. MOLECULAR CLONING: A LABORATORY MANUAL, 2^{nd} edition (1989), each of which is incorporated herein by reference in its entirety. The monoclonal antibodies of the present invention can be biologically produced by introducing an antigen such as a protein or a fragment thereof into an animal, e.g., a mouse or a rabbit. The antibody producing cells in the animal are isolated and fused with myeloma cells or heteromyeloma cells to produce hybrid cells or hybridomas.

As used herein, “nucleic acids” or “polynucleotides” refer to polymeric forms of nucleotides or analogs thereof, of any length. The polynucleotides can contain deoxyribonucleotides, ribonucleotides, and/or their analogs. Nucleotides can have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, dsRNA, and the like.

Nucleic acid molecules further include oligonucleotides, such as antisense molecules, probes, primers and the like. Oligonucleotides typically have from about 2 to about 100, 8 to about 30, or 10 to about 28 nucleotides or analogs thereof.

Nucleic acid molecules can also contain modified backbones, modified bases, and modified sugars, such as for enhancing certain desirable properties such as in vivo stability, binding affinity, etc. Modifications of nucleic acids are well known in the art and include, for example, modifications described in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361, 5,625,050, 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437, 5,677,439, 5,539,082; 5,714,331, 5,719,262, 5,489,677, 5,602,240, 5,034,506, 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633, 5,700,920, 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617, 5,681,941, 5,750,692, 5,013,830; 5,149,797; 5,220,007; 5,256,775; 5,366,878; 5,403,711; 5,491,133; 5,565,350; 5,623,065; 5,652,355; 5,652,356; and 5,700,922, each of which is incorporated herein by reference in its entirety.

Isolation, preparation, and manipulation of nucleic acids, is well known in the art and is well described in Sambrook, et al., supra.

As used herein, the term “small molecule” refers to a molecule having a molecular weight of less than about 2000 Daltons. In some embodiments, molecular weight can be about 1000, about 750, about 500, or about 300 Daltons. Small molecules can be organic, organometallic, inorganic, as well as include transition metal complexes. Suitable small molecules according to the present invention can include, for example, peptides, drugs, toxins, and the like. Drugs can include, for example, antibiotics such as beta-lactams (e.g., penicillin and amoxicillin), as well as tetracycline, ciprofloxacin, vancomycin, methicillin, erythromycin, and the like. Drugs can also include antivirals such as ribavirin, protease inhibitors (e.g., indinavir, ritonavir, etc.), nucleoside analogs (e.g., 3TC aka lamivudine, acyclovir, ganciclovir, AZT, etc.), non-nucleoside reverse-transcriptase inhibitors (e.g., efavirenz), anti-flu compounds (e.g., relenza, tamiflu, etc.), and the like.

Larger molecules such as proteins (e.g., interferons, erythropoietin, monoclonal antibodies and the like) can also be used as drugs and are often referred to as “biotherapeutics”.

“Toxins” can include, for example, botulin toxin, diphtheria toxin, anthrax toxin, cholera toxin, ricin, Clostridium difficile toxin, and the like.

The term “peptide” refers to a compound of 2 to about 50 subunit amino acids, amino acid analogs, or peptidomimetics. The subunits can be linked by peptide bonds. In other embodiments, the subunit can be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of about three or more amino acids is commonly called an oligopeptide if the peptide chain is short (e.g., under about 40 amino acids). If the peptide chain is long (e.g., more than about 40 amino acids), the peptide is commonly called a polypeptide or a protein.

As used herein, “virus” refers to any viral entity as is known in the art. Example viruses include live viral vaccines (e.g., vaccinia virus, canary pox virus, yellow fever vaccine strain 17D, recombinant alphaviruses such as venezuelan equine encephalitis, sindbis virus, etc.), gene therapy viral vectors (e.g., retroviruses, engineered retroviruses, adenoviruses, etc.), any pathogenic virus (e.g., HIV, HCV, HBV (hepB), HAV (hepA), RSV, poliovirus, smallpox virus, CMV (cytomegalovirus), flaviviruses, papillomaviruses, coronaviruses, influenza virus, etc.) Plant pathogens such as alfalfa mosaic virus, tobacco mosaic virus, etc. are also suitable.

As used herein, “multicellular organism” refers to any organism that includes differentiated cells. Example multicelluar organisms inlcude plants and animals, including mammals, such as mice, and primates, such as humans, as well as pests (e.g., agricultural pests) such as insects.

Coevolution

According to embodiments of the present invention, coevolution of a multicomponent system can be carried out by diversifying in vitro a first component of the system and diversifying in vitro a second component of the system, where the first and second component interact. Diversified populations of first and second components are created by the diversification step and typically contain a variety of different, but related, components derived from the first and second components. From these diversified populations, next generation first components and second components can be selected according to a desired criterion. In some embodiments, the selected next generation components are chosen because they exhibit one or more improved properties with respect to the original components (i.e., the first and second components before diversification or parent components).

Subsequent cycles of diversification and selection can be optionally carried out where the results of one or more previous cycles is used to influence the outcome of the subsequent cycles. For example, a selected component with improved properties over an original (parent) component can be diversified (e.g., mutated, recombined, etc.) to create a further diversified population from which a further component can be selected, having, for example, improved properties over the previously selected first component. The same steps can be carried out for the second component. This cycling can be carried out indefinitely or until a first component having a desired characteristic is identified, until a second component having a characteristic is identified, or until no further first or second components show improvement.

According to some embodiments, the methods of the present invention involve coevolving a parent target and parent neutralizing agent to produce, inter alia, improved targets or improved neutralizing agents. Coevolving, according to the present invention, includes the in vitro diversification of one or both target and neutralizing agent to produce corresponding diversified target or neutralizing agent populations having diversity, such as, for example, with respect to genotype, phenotype, molecular structure, or other feature of interest. From among the diversified populations, certain targets or neutralizing agents having desired resistance or neutralization profiles can be selected, and the process can be repeated for any number of cycles, using the selected targets or neutralizing agents as starting points in subsequent rounds of diversification and selection.

In some embodiments, coevolving involves diversifying a parent target and a parent neutralizing agent, selecting next generation neutralizing agents and targets from the respective diversified populations, where the selected neutralizing agents and targets have improved neutralizing activity or resistance (i.e., toward each other), respectively, and optionally repeating the diversifying and selecting using the next generation neutralizing agents and targets as starting points for the next cycles.

Improved neutralizing activity can be an increase in, or a newly formed, neutralizing activity against a parent or previously selected target, and improved resistance can be a newly formed resistance or increased resistance to a parent or previously selected neutralizing agent. In some embodiments, improved neutralizing activity can be neutralizing activity against a previously resistant target such as the parent target. Likewise, improved resistance can be new resistance to a neutralizing agent that previously had neutralizing activity, such as the parent neutralizing agent. The repeating can be optionally continued for one or more additional cycles or, for example, until a neutralizing agent having a desired neutralization profile is identified, until a target having a desired resistance profile is identified, or until no further neutralizing agent or targets having improved neutralizing activity or resistance can be identified.

In some embodiments, coevolving includes diversifying in vitro a parent target and a parent neutralizing agent and then selecting one or more next generation neutralizing agents and targets from populations resulting from the diversifying. The selected one or more neutralizing agents and targets can have new or improved neutralizing activity and resistance, respectively. Optionally, the diversifying and selecting can be repeated using the one or more selected next generation neutralizing agents and next generation targets in subsequent rounds of diversification and selection.

In further embodiments, coevolving includes:

i) contacting a parent neutralizing agent with members of an initial population of targets generated by diversifying a parent target in vitro;

ii) selecting one or more resistant targets within the initial population, wherein the one or more resistant targets has new or improved resistance to the parent neutralizing agent;

iii) diversifying the parent neutralizing agent in vitro to create a population of neutralizing agents;

iv) contacting members of the population of neutralizing agents with the one or more resistant targets selected in step ii);

v) selecting one or more further neutralizing agents from the population of neutralizing agents, wherein the one or more further neutralizing agents has new or improved neutralizing activity against the one or more resistant targets;

vi) optionally repeating steps i) and ii) or i), ii), iii), iv), and v). In repeating the process, the one or more further neutralizing agents can be used in place of the parent neutralizing agent and the one or more resistant targets can be used in place of the parent target.

Diversification of a target or neutralizing agent can be achieved by any suitable in vitro method that can create a population of related targets or neutralizing agents. For example, diversification of a nucleic acid target can be carried out by any of numerous known methods such as mutagenesis, gene shuffling, and the like to generate a population of different but related nucleic acids. The members of the population of nucleic acids can be related by, for example, homology as can be measured by percent identity (BLAST with default parameters). In some embodiments, members of a population of nucleic acids have greater than about 50, 60, 70, 80, 90, 95, or 99% identity. According to another example, the structure of a small molecule can be varied by any of numerous known methods including combinatorial synthesis to generate a population of different but related small molecules. For example, members of the population of small molecules can share a selected core structure or other structural or functional feature while having variable substituents.

Mutagenesis methods for diversifying cells, bacteria, nucleic acids, proteins, and the like are well described in the literature. Mutagenesis methods include error prone PCR Leung et al., Technique, 1989, 1, 11-15, use of mutagenic strains such as the XL1-Red mutator strain of E. coli (Stratagene Inc.), use of random mutagenesis methods involving mutagenic chemicals such as ethyl-methyl sulfonate (EMS) or involving irradiation by UV light or other radiations of higher or lower energy, combinatorial cassette mutagenesis (Delagrave et al., Protein Eng., 1993, 6, 327-331; Delagrave, et al., Bio/Technology, 1993, 10, 1548-52), site-directed mutagenesis, mutagenesis by PCR involving the incorporation of one or more primers encoding mutations, mutagenesis by DNA shuffling (e.g., Stemmer, Nature, 1994, 370, 389; and U.S. Pat. Nos. 5,830,721; 5,811,238; and 5,605,793), and mutagenesis by any PCR method.

Further methods for diversification of cells include mutating a gene or other nucleic acid, for example, by any of the above methods, and introducing these mutated nucleic acids into cells by known methods. Another method is sequential random mutagenesis (SRM), in which various strains of microorganisms are improved via mutagenesis of their genomes or episomes and screening. Mutagenesis methods used in SRM are described in Zhang et al., Nature, 2002, 415, 644; Aharonowitz & Cohen, Sci. Am., 1981, 245, 141; Demain & Solomon, Eds., Manual of Industrial Microbiology and Biotechnology, (ASM, Washington, 1986); and Vinci & Byng in Manual of Industrial Microbiology and Biotechnology (eds Demain & Davie) 103-113 (ASM, Washington, 1999). Cells, such as pathogenic microorganisms, can be diversified by taking advantage of their natural mutation rates and merely culturing them in vitro to create a population of closely related cells differing from each other by one or a few mutations.

Methods for diversification of viruses include, for example, use of recombinant DNA technologies to make infectious molecular clones of a viral genome. This technique enables the introduction of mutations in such genomes using site-directed mutagenesis (Monath et al., Vaccine, 1999, 17, 1869) and PCR-based mutagenesis methods (Gritsun & Gould, Virology, 1995, 214, 611). Certain viruses, particularly those whose genomes are made of RNA have high inherent mutation rates such that merely culturing them in vitro results in so called quasispecies: a population of closely related viruses differing from each other by one or a few mutations.

Methods for diversification of small molecules include combinatorial synthetic methods such as, for example, attachment of combinatorial peptide libraries to small molecule antibiotics as described by Li and Roberts, Chem. & Biol., 2003, 10, 233. Other examples include the use of solid phase synthesis methods to prepare libraries of derivatives of small molecule drugs like vancomycin (Nicolaou, et al., Chem. Eur. J., 2001, 7, 3798), and related combinatorial methods reviewed by Breinbauer, et al., Curr. Med. Chem., 2002, 9, 2129. Further methods include combinatorial biosynthesis whereby polyketide synthetase complexes are genetically engineered to produce libraries of related compounds such as erythromycin analogues (Cane, et al., Science, 1998, 282, 63; Khosla, J. Org. Chem, 2000, 65, 8127; and McDaniel, et al., Proc. Natl. Acad. Sci. USA, 1999, 96, 1846).

Selection of population members can be carried out by any of numerous methods known in the art. Typically, selection is carried out by screening a population for a desired characteristic such as neutralizing activity, resistance, antigenic activity, catalytic activity, binding affinity, biological phenotype, and the like.

Selection methods for populations of cells and bacteria include growth in the presence of a drug according to methods well known in the art. Cells can also be selected by means of Fluorescence Activated Cell Sorting (FACS; e.g., Fu et al, Nat. Biotechnol., 1999, 17, 1109). Cells can further be selected by incubation in the presence of selective growth media containing toxins, antibiotics, or competing organisms.

Selection methods for populations of nucleic acids can include any selection method known in the art. For example, catalytic RNAs can be selected according to procedures set out in Joyce, Gene, 1989, 82, 83. Nucleic acids binding to proteins or other ligands can be selected by SELEX as described, for instance, in Vo et al, Virology, 2003, 307, 301.

Selection methods for proteins are numerous in the art, for example, phage display methods which select recombinant phage (bacterial viruses) for the ability to bind certain ligands via the recombinant proteins they express on their surface (see, e.g., Barbas et al., Proc. Natl. Acad. Sci. USA, 1994, 91, 3809). Other selection methods for proteins are reported in, e.g., (Joo et al., Chem. Biol., 1999, 6, 699; Joo et al., Nature, 1999, 399, 670; Miyazaki et al., J. Mol. Evol., 1999, 49, 716; Chen et al., Proc. Natl. Acad. Sci. USA, 1993, 90, 5618; Chen et al., Biotechnology, 1991, 9, 1073; You et al., Protein Eng., 1996, 9, 77; Marrs et al., Curr. Opin. Microbiol., 1999, 2, 241; U.S. Pat. No. 5,914,245; and U.S. Pat. No. 5,283,173.)

Selection methods for viruses include, growth in the presence of an antiviral compounds such as an antibody is described by Crowe et al., Virology, 1998, 252, 373.

Selection methods for small molecules include screens for effectiveness in an appropriate biological assay such as antibiotic activity according to known methods. Electrospray (ES) or matrix assisted laser desorption ionization (MALDI) mass spectrometry can be used to screen for small molecules capable of binding a specific target protein. High-throughput enzyme inhibition assays are frequently used in the pharmaceutical industry to identify small molecules that could be useful as pharmaceuticals (e.g., Hamilton et al, Protein Sci., 2003, 12, 458).

In some embodiments, diversification and selection can be carried out simultaneously in the coevolution process. For example, microorganisms having a natural mutation rate can be grown in the presence of an agent that selects for resistant mutants.

Use of the selected targets and neutralizing agents in subsequent rounds of diversification can be carried out by any of known methods in the art. As discussed above, information derived from the selected targets and neutralizing agents can help generate subsequent populations having increased probability of producing a target or neutralizing agent with properties that are improved compared with a parent or a selected target or neutralizing agent from a previous selection step. For example, in the case where diversifying of nucleic acids and proteins is carried out by recombinant methods such as shuffling, use of selected targets or neutralizing agents to prepare further populations can increase the chances of finding a further improved molecule because the “successful” sequence of the selected molecules will continue to be propagated and can even be emphasized or “weighted.” Similarly, in the case where diversifying is carried out by combinatorial synthesis of chemical compounds, features of selected compounds (e.g., type and/or position of certain functional groups) can be retained or emphasized in subsequent rounds of diversification.

In some embodiments of the invention, preparation of populations of target or neutralizing agent in addition to the populations derived from the parent molecules are not generated using selected targets or neutralizing agents. Instead, populations generated from the parent molecule can be sampled one or more additional times. For example, after a resistant target is selected, further neutralizing agents can be selected from the population generated from the parent neutralizing agent instead of from a subsequently generated population.

According to some embodiments of the present invention, the selected neutralizing agents and/or targets from each diversification and selection cycle can form a “collection” of evolved neutralizing agents or a “collection” of evolved targets, respectively. These collections can also include parent neutralizing agents and targets used in an initial cycle of the coevolution process. The members of these collections can represent some of the most improved neutralizing agents or targets for their generation, and/or represent the neutralizing agents or targets closest to a certain neutralization or resistance profile for their generation.

Desired neutralization or resistance profiles can be defined by the breadth and/or potency of neutralizing activity or resistance. Breadth of neutralization can be determined by the number of different targets that a particular neutralizing agent shows whole and/or partial neutralizing activity toward. The more targets neutralized by a particular neutralizing agent, the broader the neutralizing activity. Thus, a desired neutralization profile can represent a certain breadth of neutralization activity, measured, for example, by a percentage of members of the collection of evolved targets susceptible to the neutralization effects of a neutralizing agent. In some embodiments, a desired neutralization profile is neutralization activity against the greatest number of members of a population of targets. In other embodiments, a desired resistance profile is resistance to the greatest number of members of a population of neutralizing agents.

Potency of neutralization can be determined by the relative strength of neutralizing activity. For example, serially diluted neutralizing agent can be tested against a fixed amount of target. The concentration of neutralizing agent at which 50% of the target is neutralized is the EC₅₀ (effective concentration 50%). Neutralizing agents with greater potency have lower EC₅₀ values. Thus, a desired neutralization profile can be a certain neutralizing potency such as a threshold neutralizing potency, or a greatest potency. In some embodiments, neutralizing potency can be an EC₅₀ value less than about 10,000, less than about 1000, less than about 500, less than about 100, or less than about 50 nM.

Often, a desired neutralization profile can be a combination of breadth and potency. In some embodiments, desired neutralization profile can be neutralizing activity against a previously resistant target. Similarly, a desired resistance profile can be resistance to a previously neutralizing neutralizing agent.

Members of collections can be cross-tested (e.g., testing neutralizing agents against targets) to identify a collection member having a certain characteristic, such as, for example, to determine breadth of neutralizing activity or resistance for a particular collection member. For example, all or select members of a collection of neutralizing agents can be tested for neutralizing activity against all of or a select number of members of a collection of targets. As an example, a three-member collection of antibodies (neutralizing agent) can be formed from the antibodies selected during the selection step of a coevolution process. Similarly, a three-member collection of pathogens (targets) can be formed in the same manner. The three members of the collection of antibodies can each be tested for neutralizing activity against each of the three members of the collection of pathogens. An antibody showing neutralizing activity against the most number of members in the collection of pathogens can be identified as having the broadest neutralizing activity. Conversely, a pathogen from the collection of pathogens showing resistance to the most number of antibodies can be identified as having the broadest resistance.

Accordingly, the present invention further provides methods of preparing a neutralizing agent having a desired neutralizing profile or a target having a desired resistance profile comprising:

a) coevolving a parent target and a parent neutralizing agent to generate a collection of evolved neutralizing agents and a collection of evolved targets;

b) cross testing members of the collections for neutralizing activity or resistance;

c) identifying at least one member of the collection of evolved neutralizing agents having the desired neutralizing profile or identifying at least one member of the collection of evolved targets having the desired resistance profile.

In further embodiments, methods of the present invention can further include diversifying a target or neutralizing agent identified after cross-testing and using the resulting diversified population in one or more further coevolution cycles.

The present invention further provides methods of preparing neutralizing agents having a desired neutralization profile comprising:

a) diversifying a parent neutralizing agent to create a population of diversified neutralizing agents;

b) testing the members of the population of diversified neutralizing agents for neutralizing activity against members of a population of targets prepared by in vitro diversification of a parent target; and

c) identifying at least one diversified neutralizing agent from step a) having the desired neutralization profile.

d) optionally, repeating steps a) through c).

Conversely, the present invention further provides methods of preparing a target having a desired resistance profile comprising:

a) diversifying a parent target to create a population of diversified targets;

b) testing the members of the population of diversified targets for resistance to members of a population of neutralizing agents prepared by diversification of a parent neutralizing agent; and

c) identifying at least one diversified target from step a) having the desired resistance profile.

d) optionally, repeating steps a) through c).

An example method according to the present invention includes a system having two members, such as a single-chain fragment of a monoclonal antibody and the respiratory syncytial virus (RSV), a human pathogen that can be neutralized by this antibody fragment. Mutant viruses resistant to antibody neutralization can be selected in tissue culture. Antibody variants capable of neutralizing these mutant viruses can be generated via mutagenesis and phage display. This process can be carried out iteratively, resulting in a number of coevolved antibody variants which can be tested for their ability to suppress the evolution of antibody-resistant viruses in tissue culture.

Therapeutic and Prophylactic Methods

Methods of treatment according to the present invention can include both prophylaxis and therapy. Prophylaxis or therapy can be accomplished by administration of therapeutic agents such as targets or neutralizing agents prepared by the coevolution methods described herein. In some embodiments, methods of treatment include administration of an antibody. In other embodiments, methods of treatment include administration of an antigen. The therapeutic agent can be administered at a single time point or multiple time points to a single or multiple sites. Administration can also be nearly simultaneous to multiple sites. Patients or subjects include mammals, such as human, bovine, equine, canine, feline, porcine, and ovine animals. The subject is preferably a human.

A disease or disorder, such as a viral infection, cancer, allergy, or other pathological condition associated with a target, can be diagnosed using criteria generally accepted in the art, including, for example, the presence of a malignant tumor or elevated white blood cell count. Therapeutic agents can be administered either prior to or following surgical removal of primary tumors and/or treatment such as administration of radiotherapy or conventional chemotherapeutic drugs. In further embodiments, therapeutic agents such as vaccines can also be administered prior to infection by an infectious agent such as a virus, bacteria, or other pathogen.

Within certain embodiments, therapy can be immunotherapy, which can be active immunotherapy in which treatment relies on the in vivo stimulation of the endogenous host immune system to react against tumors or infected cells with the administration of antigens prepared according to the methods described herein. Within other embodiments, immunotherapy can be passive immunotherapy, in which treatment involves the delivery of agents with, for example, immune reactivity (such as effector cells or antibodies) that can directly or indirectly mediate antitumor, anti-inflammatory, or other effects and do not necessarily depend on an intact host immune system. Examples of effector cells include T cells, T lymphocytes (such as CD8+ cytotoxic T lymphocytes and CD4+ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein.

The therapeutic agents prepared according to the coevolution methods described herein can be combined with a pharmaceutically acceptable carrier to produce a pharmaceutical composition. As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers ate formulated by well known conventional methods (see, for example, Remington's Pharmaceutical Sciences, Chapter 43, 14th Ed., Mack Publishing Co, Easton Pa. 18042, USA).

Therapeutic and Prophylactic Compositions and Uses of Antibodies

Antibodies and antibody fragments can be useful in the treatment of numerous disorders including, for example, cancer, inflammatory disorders, such as adult respiratory distress syndrome (ARDS), hypovolemic shock, ulcerative colitis, rheumatoid arthritis, and others, as shown in Table 0.

TABLE 0
Monoclonal antibody-based therapeutics
Nature Biotechnology, 2003, 21, 868.

| | | | Year |
|---|---|---|---|
| Product | Company | Indication | approved |
| Bexxar (tositumomab; | Corixa/GlaxoSmith | Treatment of CD20 positive | 2003 (US) |
| radiolabelled monoclonal | Kline | follicular non-Hodgkin | |
| antibody directed against | | lymphoma | |
| CD20, produced in a | | | |
| mammalian cell line.) | | | |
| Xolair (Omalizumab; | Genentech | Asthma | 2003 (US) |
| rIgG1k Mab that binds | | | |
| IgE, produced in CHO | | | |
| cells) | | | |
| Humira (adalimumab; r | Abbott Laboratories | Rheymatoid arthritis | 2002 (US) |
| human Mab (antiTNF) | | | |
| created using phage | | | |
| display technology and | | | |
| produced in a mammalian | | | |
| cell line) | | | |
| Zevalin (Ibritumomab | IDEC | Non-Hodgkin lymphoma | 2002 (US) |
| Tiuxetan; murine Mab | Pharmaceuticals | | |
| produced in a CHO cell | | | |
| line, targeted against the | | | |
| CD20 antigen. A | | | |
| radiotherapy agent.) | | | |
| Mabcampath (EU) or | Millennium & | Chronic lymphocytic | 2001 (EU, |
| Campath (US) | ILEX (EU); Berlex, | leukemia | US) |
| (alemtuzumab; a | ILEX & | | |
| humanized monoclonal | Millennium | | |
| antibody directed against | Pharmaceuticals | | |
| CD52 surface antigen of | (US) | | |
| B-lymphocytes.) | | | |
| Mylotarg (gemtuzumab | Wyeth | Acute myeloid leukemia | 2000 (US) |
| zogamicin; a humanized | | | |
| antibody-toxic antibiotic | | | |
| conjuage targeted against | | | |
| CD33 antigen found on | | | |
| leukemic blast cells.) | | | |
| Herceptin (trastuzumab, | Genentech (US); | Treatment of metastatic | 1998 (US), |
| humanized antibody | Roche (EU) | breast cancer if tumor | 2000 (EU) |
| directed against human | | overexpresses HER2 protein | |
| epidermal growth factor | | | |
| receptor 2 (HER2)) | | | |
| Remicade (infliximab, | Centocor | Treatment of Crohn disease | 1998 (US), |
| chimeric mAb directed | | | 1999 (EU) |
| against TNF-alpha | | | |
| Synagis (palivizumab, | MedImmune (US); | Prophylaxis of lower | 1998 (US), |
| humanized mAb directed | Abbott (EU) | respiratory disease caused by | 1999 (EU) |
| against an epitope on the | | syncytial virus in pediatric | |
| surface of respiratory | | patients | |
| syncytial virus.) | | | |
| Zenapax (daclizumab, | Hoffmann-La | Prevention of acute kidney | 1997 (US), |
| humanized mAb directed | Roche | transplant rejection | 1999 (EU) |
| against the alpha-chain of | | | |
| the IL-2 receptor) | | | |
| Humaspect (Votumumab, | Organon Teknika | Detection of carcinoma of | 1998 (EU) |
| human mAb directed | | the colon or rectum | |
| against cytokeratin tumor- | | | |
| associated antigen) | | | |
| Mabthera (Rituximab, | Hoffmann-La | Non-Hodgkin lymphoma | 1998 (EU) |
| chimeric mAb directed | Roche | | |
| against CD20 surface | | | |
| antigen of B lymphocytes. | | | |
| See also Rituxan.) | | | |
| Simulect (basiliximab, | Novartis | Prophylaxis of acute organ | 1998 (EU) |
| chimeric mAb directed | | rejection in allogeneic renal | |
| against the alpha-chain of | | transplantation | |
| the IL-2 receptor) | | | |
| LeukoScan (Sulesomab, | Immunomedics | Diagnostic imaging for | 1997 (EU) |
| murine mAb fragment | | infection/inflammation in | |
| (Fab) directed against | | bone of patients with | |
| NCA 90, a surface | | osteomyelitis | |
| granulocyte nonspecific | | | |
| cross-reacting antigen.) | | | |
| Rituxan (rituximab | Genentech/IDEC | Non-Hodgkin lymphoma | 1997 (US) |
| chimeric mAb directed | Pharmaceuticals | | |
| against CD20 antigen | | | |
| found on the surface of B | | | |
| lymphocytes) | | | |
| Verluma (Nofetumomab | Boehringer | Detection of small-cell lung | 1996 (US) |
| murine mAb fragments | Ingelheim/NeoRx | cancer | |
| (Fab) directed against | | | |
| carcinoma-associated | | | |
| antigen.) | | | |
| Tecnemab KI (murine | Sorin | Diagnosis of cutaneous | 1996 (EU) |
| mAb fragments (Fab/Fab2 | | melanoma lesions | |
| mix) directed against | | | |
| HMW-MAA) | | | |
| ProstaScint (capromab- | Cytogen | Detection/staging/follow-up | 1996 (US) |
| pentetate, murine mAb | | of prostate adenocarcinoma | |
| directed against the tumor | | | |
| surface antigen PSMA) | | | |
| MyoScint (imiciromab- | Centocor | Myocardial infarction | 1996 (US) |
| pentetate, murine mAb | | imaging agent | |
| fragment directed against | | | |
| human cardiac myosin) | | | |
| CEA-scan (arcitumomab, | Immunomedics | Detection of | 1996 (US, |
| murine mAb fragment | | recurrent/metastatic | EU) |
| (Fab), directed against | | colorectal cancer | |
| human carcinoembryonic | | | |
| antigen, CEA) | | | |
| Indimacis 125 (Igovomab, | CIS Bio | Diagnosis of ovarian | 1996 (EU) |
| murine mAb fragment | | adenocarcinoma | |
| (Fab2) directed against the | | | |
| tumor-associated antigen | | | |
| CA 125) | | | |
| ReoPro (abciximab, Fab | Centocor | Prevention of blood clots | 1994 (US) |
| fragments derived from a | | | |
| chimeric mAb, directed | | | |
| against the platelet surface | | | |
| receptor GPIIb/IIIa) | | | |
| OncoScint CR/OV | Cytogen | Detection/staging/follow-up | 1992 (US) |
| (satumomab pendetide, | | of colorectal and ovarian | |
| murine mAb directed | | cancers | |
| against TAG-72, a tumor- | | | |
| associated glycoprotein) | | | |
| Orthoclone OKT3 | Ortho Biotech | Reversal of acute kidney | 1986 (US) |
| (Muromomab CD3, | | transplant rejection | |
| murine mAb directed | | | |
| against the T-lymphocyte | | | |
| surface antigen CD3) | | | |

Therapeutic formulations of antibodies and antibody fragments can be prepared for storage by mixing the antibody or antibody fragment having the desired degree of purity with optional pharmaceutically acceptable carriers, excipients, or stabilizers (see, e.g., Remington's Pharmaceutical Sciences, supra), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

Antibodies or antibody fragments for in vivo administration are preferably sterile. This is readily accomplished by filtration through sterile filtration membranes, prior to or following lyophilization and reconstitution. The antibodies or antibody fragments ordinarily will be stored in lyophilized form or in solution.

Therapeutic antibody or antibody fragment compositions generally are placed into a container having a sterile access port, for example, an intravenous solution bag or vial having a stopper pierceable by a hypodermic injection needle.

The route of antibody or antibody fragment administration can be carried out in accord with known methods, e.g., inhalation, injection or infusion by intravenous, intraperitoneal, intracerebral, intramuscular, intraocular, intraarterial, or intralesional routes, by enema or suppository, or by sustained release systems as noted below. Preferably the antibody is given systemically or at a site of inflammation.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919 and EP 58,481), copolymers of L-glutamic acid and gamma ethyl-L-glutamate (Sidman et al., Biopolymers, 1983, 22, 547), poly (2-hydroxyethyl-methacrylate) (Langer et al., J. Biomed. Mater. Res., 1981, 15, 167 and Langer, Chem. Tech., 1982, 12, 98), ethylene vinyl acetate (Langer et al., supra) or poly-D-(−)-3-hydroxybutyric acid (EP 133,988). Sustained-release compositions also include liposomally entrapped antibody or antibody fragment. Liposomes containing an antibody or antibody fragment can be prepared by methods known per se: DE 3,218,121; Epstein et al., Proc. Natl. Acad. Sci. U.S.A., 1985, 82, 3688; Hwang et al., Proc. Natl. Acad. Sci. U.S.A., 1980, 77, 4030; EP 52,322; EP 36,676; EP 88,046; EP 143,949; EP 142,641; U.S. Pat. Nos. 4,485,045 and 4,544,545; and EP 102,324. Ordinarily the liposomes are of the small (about 200-800 Angstroms) unilamelar type in which the lipid content is greater than about 30 mole percent cholesterol, the selected proportion being adjusted for the most efficacious antibody or antibody fragment therapy.

An “effective amount” of the antibody or antibody fragment to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect. Typically, the clinician will administer the antibody or antibody fragment until a dosage is reached that achieves the desired effect. The progress of this therapy is easily monitored by conventional assays.

In the treatment and prevention of a disease or disorder with an antibody or antibody fragment of the invention, the antibody composition can be formulated, dosed, and administered in a fashion consistent with good medical practice. Factors for consideration in this context include the particular disorder being treated, the particular mammal being treated, the clinical condition of the individual patient, the cause of the disorder, the site of delivery of the antibody, the particular type of antibody, the method of administration, the scheduling of administration, and other factors known to medical practitioners. The “therapeutically effective amount” of antibody to be administered can be governed by such considerations, and is the minimum amount necessary to prevent, ameliorate, or treat the inflammatory disorder. Such amount is preferably below the amount that is toxic to the host or renders the host significantly more susceptible to infections.

As a general proposition, the initial pharmaceutically effective amount of the antibody or antibody fragment administered parenterally per dose can be in the range of about 0.1 to 50 mg/kg of patient body weight per day, with the typical initial range of antibody used being 0.3 to 20 mg/kg/day, more preferably 0.3 to 15 mg/kg/day. As noted above, however, these suggested amounts of antibody or antibody fragment are subject to therapeutic discretion.

The antibody or antibody fragment need not be, but is optionally formulated with one or more agents currently used to prevent or treat the disease or disorder in question. For example, in rheumatoid arthritis, an antibody can be given in conjunction with a glucocorticosteroid, or for cancer, an antibody can be given in conjunction with a chemotherapeutic. The antibody or antibody fragment can also be formulated with one or more other antibodies or antibody fragments to provide a therapeutic antibody “cocktail.”

Therapeutic and Prophylactic Compositions of Antigens

The invention provides preventative antigens such as, for example, peptides, proteins, antigenic nucleic acids, and T cells or other antigen presenting cells and the like that can be incorporated into immunogenic compositions (i.e., vaccines). Pharmaceutical compositions can comprise one or more such components and, optionally, a physiologically acceptable carrier. Vaccines can comprise one or more such components and optionally an adjuvant that serves as a non-specific immune response enhancer. The adjuvant can be any substance that enhances an immune response to an exogenous antigen. Examples of adjuvants include conventional adjuvants, biodegradable microspheres (e.g., polylactic galactide), immunostimulatory oligonucleotides and liposomes (into which the compound is incorporated; see e.g., Fullerton, U.S. Pat. No. 4,235,877). Vaccine preparation is generally described in, for example, M. F. Powell and M. J. Newman, eds., Vaccine Design (the subunit and adjuvant approach), Plenum Press (NY, 1995). Pharmaceutical compositions and vaccines within the scope of the present invention can also contain other compounds that may be biologically active or inactive. For example, one or more immunogenic portions of other antigens may be present, either incorporated into a fusion polypeptide or as a separate compound, within the composition or vaccine.

In other embodiments, a pharmaceutical composition or vaccine can contain nucleic acids encoding one or more of the antigens, such that the antigen is generated in situ. As noted above, the nucleic acid can be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems, 1998, 15, 143. Appropriate nucleic acid expression systems contain the necessary nucleic acid sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope.

The dosage of vaccine administered to a patient, in the context of the present invention, should be sufficient to effect a beneficial therapeutic response in the patient over time, or to inhibit onset of the disease or disorder or inhibit disease or disorder progression. Thus, the composition can be administered to a subject in an amount sufficient to elicit an effective immune response to the specific antigens and/or to alleviate, reduce, cure or at least partially arrest symptoms and/or complications from the disease or disorder. An amount adequate to accomplish this is included in the term a “therapeutically effective amount.”

Routes and frequency of administration of the therapeutic agents and compositions disclosed herein, as well as dosage, can vary from individual to individual, and can be readily established using standard techniques. In general, the pharmaceutical compositions and vaccines can be administered, by injection (e.g., intracutaneous, intratumoral, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients.

A suitable dose is an amount of an antigen that, when administered, is capable of promoting an immune response, and is at least 10-50% above the basal (i.e., untreated) level. Such response can be monitored, for example, by measuring the antibodies in a patient or by vaccine-dependent generation of cytolytic effector cells capable of killing the patient's infected cells or tumor cells in vitro. Such vaccines can also be capable of causing an immune response that leads to an improved clinical outcome (e.g., more frequent remissions for cancer patients, complete or partial or longer disease-free survival) in vaccinated patients as compared to nonvaccinated patients. In general, for pharmaceutical compositions and vaccines comprising one or more antigens, the amount of each antigen present in a dose ranges from about 100 μg to 5 mg per kg of host. Suitable volumes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor or other disease-related protein generally correlate with an improved clinical outcome. Such immune responses can be evaluated using standard proliferation, cytotoxicity or cytokine assays, which can be performed using samples obtained from a patient before and after treatment.

Additional Methods

The invention further provides a method for detecting a disease or disorder such as cancer in a sample, comprising contacting the sample with a molecule, such as an antibody, that binds to a target in the sample, where the target is associated with the disease or disorder. The target can be, for example, a nucleic acid or protein encoded thereby. The sample can be tissue from a mammal, such as human, bovine, equine, canine, feline, porcine, and ovine tissue. In some embodiments, the tissue is human. The tissue can comprise a tumor specimen, cerebrospinal fluid, or other suitable specimen. In one embodiment, the method comprises use of an ELISA type assay that employs an antibody by the coevolution methods described herein to detect the presence of target in a specimen. This method can also be used to monitor target levels in a tissue sample of a patient. For example, the suitability of a therapeutic regimen for initial or continued treatment can be determined by monitoring target levels according to this method.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. These methods are described in the following publications. See, e.g., Sambrook, et al., Molecular Cloning: A Laboratory Manual, 2nd edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson et al., eds. (1995)); Antibodies, A Laboratory Manual (Harlow and Lane eds. (1988)); Animal Cell Culture (R. I. Freshney ed. (1987)); and Phage Display: A Laboratory Manual (C.F. Barbas III et al., (2001)), each of which is incorporated herein by reference in its entirety.

The following examples are presented to illustrate the present invention and to assist one of ordinary skill in making and using the same. The examples are not intended in any way to otherwise limit the scope of the invention.

### EXAMPLES

### Example 1

### Coevolution of a Monoclonal Antibody/Pathogen System

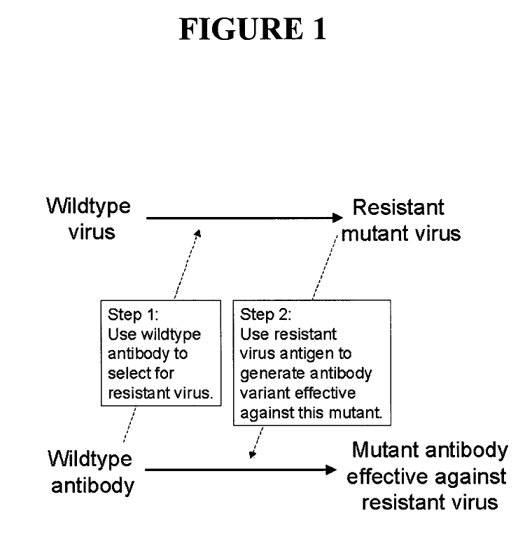

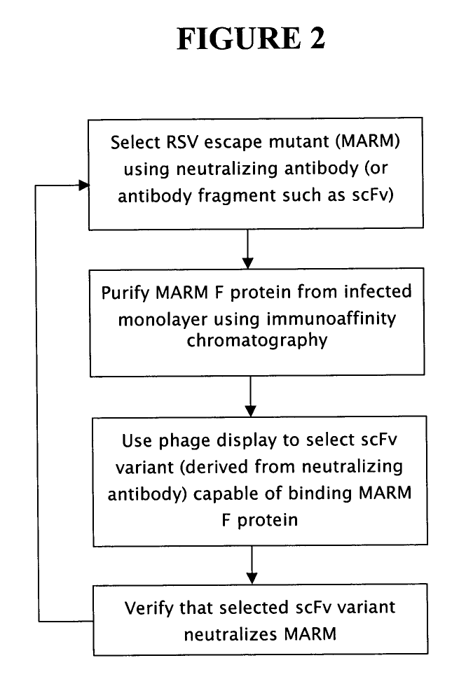

This example shows how coevolution can be used to develop monoclonal antibody-resistant mutants (MARMs) of respiratory syncytial virus (RSV) and evolve antibodies capable of neutralizing these MARMs. FIG. 1 shows a two-step coevolution scheme where wildtype virus is grown with ‘wildtype’ antibody to select resistant strains (MARMS). Antigen from the resulting resistant virus can be used to generate, e.g., via in vitro evolution techniques, an antibody which overcomes this resistance. FIG. 2 gives an outline of an example coevolution scheme.

Selecting MARMs

MARMs can be isolated using techniques described previously, for example, by Crowe, et al., Virology, 1998, 252, 373; Lopez, et al., J. Virol., 1998, 72, 6922; Garcia-Barreno, J. Virol., 1998, 72, 6922; Garcia-Barreno, J. Virol., 1989, 63, 925; and Lopez, et al. J. Virol., 1990, 64, 927. Equal volumes of RSV Long (4×10⁶ pfu/mL, obtained from ATCC and amplified in-house on HEp-2 cells, also from ATCC) and 200 μM HNK20 antibody diluted in culture medium (DMEM or MEME supplemented with 2.5% Fetal Bovine Serum: DMEM2.5) are mixed. The mixture is incubated at 37° C. for 60 minutes. Then 0.15 mL of the resulting mixture is added to a monolayer of HEp-2 cells in one well of a 24-well plate and allowed to infect the monolayer for 2 hours. The inoculum is removed and replaced with 1 mL of medium (DMEM2.5). Several replicates can be prepared in the same plate. A control sample is included in which irrelevant antibody is added to RSV (mock neutralization). After 3 to 4 days, significant cytopathic effects (cpe) can be seen in the mock neutralization sample but not in the neutralized samples. A 75 μL aliquot of the culture supernatant from a neutralized sample is then mixed with an equal volume of 200 μM HNK20 antibody, as above, and the above steps are repeated. Several cycles of this process may be required before cpe is observed in samples other than the mock neutralization control (see Table 1, # of passages). Selection of MARMs can be from RSV Long (e.g., the prototypical subgroup A strain), as well as other strains such as A-2, and 18537 (prototypical subgroup B strain). It has already been shown that HNK20 neutralizes these three strains and numerous clinical isolates.

Once cpe is observed, MARMs are plaque-purified. The culture supernatant in the well (or wells) showing cpe is used to infect a HEp-2 monolayer in a 35 mm plate. After adsorption, DMEM2.5 containing 0.7% agar is used as an overlay and plates are incubated 5 days (37° C.) until a second overlay containing neutral red is added and incubation is continued for an additional day. Plaques are picked and an additional round of plaque purification is carried out to ensure clone purity. Virus eluted from the final plaques is assayed to verify lack of sensitivity to the neutralizing antibody. Microneutralization assays and plaque-reduction neutralization tests (PRNT) can be performed as described in, for example, Delagrave, et al., Prot. Eng., 1999, 12, 357. Polyclonal neutralizing serum and other anti-RSV monoclonal antibodies (BioDesign, Saco, M E) can be used as positive controls of neutralization.

Once isolated, MARMs can be characterized with respect to the DNA sequence of their F gene. RSV MARMs are grown in duplicate to high titer (>10⁶ p fu/mL, achieved routinely in our lab) on HEp-2 cells using DMEM+10% FBS. Qiagen viral RNA purification kit is used to isolate viral genomic RNA from the culture supernatants. One-step RT-PCR is carried out in duplicate to amplify the F gene. Depending on the amplification target's length and composition, two-step RT-PCR occasionally performs more reliably and can be considered as an alternative to one-step RT-PCR if necessary. Both strands of the duplicate PCR products are then sequenced directly (following routine purification using Qiagen PCR purification kit) using dye-terminator dideoxy sequencing implemented on an Applied Biosystems sequencing platform offered by local sequencing services (e.g., Allan Laboratories DNA sequencing service offered by the University of Delaware.) Sequences are analyzed using the “DNAStar” suite of computer programs or similar software. Any discrepancies between duplicate samples are resolved by additional culturing and sequencing. The original RSV strain used to select MARMs is sequenced as a control.

TABLE 1
Results of RSV MARM selection experiments from the literature.

| Antibody | | | Amino acid | |
|---|---|---|---|---|
| name | # of passages | # of MARMs^{a} | substitutions^{b} | Reference^{c} |
| 7.936 | 12 to 20 | 4 (3) | V447A (×2) | Lopez 1 |
| | | | K433T (×1) | |
| | | | I432T (×1) | |
| 9.432 | 12 to 20 | 3 (1) | S436F (×3) | Lopez 1 |
| 47F | 3 | 5 (2) | N272Y (×2) | Lopez 2 |
| | | | N268I (×3) | |
| Fab 19 | 2 | 3 (1) | I266M (×3) | Crowe |

^{a}Number of MARMs selected and, shown in brackets, number of unique MARMs.
^{b}Number of times a particular substitution was found is indicated in brackets.
^{c}References: Lopez 1: Lopez, et al., J. Virol., 1998, 72, 6922; Lopez 2: Lopez, et al., J. Virol., 1990, 64, 927; Crowe: Crowe, et al., Virology, 1998, 252, 373.

The scientific literature provides guidance as to the expected results from the above procedure. Table 1 indicates that 1 to 3 unique MARMs are typically observed in published antibody-resistance selections of RSV. Each such MARM usually shows a single amino acid substitution.

Selected MARMs are grown on HEp-2 monolayers to provide antigen (F protein) to be used in the selection of antibody fragments capable of neutralizing the MARMs. Antigen used for affinity selection can be produced as described in Calder, et al., Virology, 2000, 122, although different monoclonal antibodies may be used for affinity purification, for example as described in Guirakhoo, et al., Immunotechnology, 1996, 2, 219. HEp-2 cells are infected with 0.1 MOI (multiplicity of infection) of RSV MARM and allowed to incubate for 3 days, at which point viral titers reach ˜4×10⁶ pfu/mL and cytopathic effects are pronounced. (This assumes wildtype-like replication. Adjustments may be required if the MARM behaves differently.) Cells and cell debris are recovered by centrifugation, washed in 1×PBS and resuspended in lysis buffer containing detergent (Calder, et al., Virology, 2000, 122). Insoluble cell debris is then removed by centrifugation; soluble cell extract containing RSV F protein is harvested and subjected to affinity chromatography to purify F protein (Calder, et al., Virology, 2000, 122). An ELISA can be performed to verify that the antigen is not wildtype: it will presumably not be recognized by the antibody used to select for the MARM, or at least not as well. Also, sequencing of partial viral genomic cDNA isolated from the culture supernatant can be used to ensure that the MARM replicated successfully, without wildtype or other contaminating strains.

More than one MARM can be selected by the above procedure (Table 1). In order to develop a robust drug, one can select new antibodies (per the procedures discussed below) against each of the selected MARMs.

Overcoming Resistance

In vitro evolution of scFv antibody fragments can be carried out in a manner that will not only identify variants capable of binding mutant antigens with high affinity, but will also ensure that neutralization potency is maintained or improved compared to the parental scFv. High-throughput affinity selection methods such as phage display are convenient means of rapidly selecting for antibody fragments recognizing a specific antigen (see, e.g., Gram, et al., Proc. Natl. Acad. Sci. USA, 1992, 89, 3576. Therefore, phage display can be used to select scFv variants capable of binding to the MARM F protein. HNK20 scFv has already been successfully phage displayed. Prior to affinity panning, however, libraries of mutant HNK20 scFv genes can be cloned into the phage display vector

Three scFv mutant libraries can be prepared and pooled: one created by error-prone PCR (Leung, et al., Technique, 1989, 1, 11)., one combinatorial library in which the first five residues of HCDR3 (H94 to H98, Kabat numbering—see FIG. 2 in Delagrave, et al., 1999, supra) are randomly mutated, and a second combinatorial library in which the last five residues of HCDR3 (H99 to H102) are randomly mutated. HCDR3 is chosen because it is generally regarded as the principal determinant of antibody specificity. Two libraries are used to span HCDR3 because it would be difficult to make a comprehensive phage display library spanning all 10 residues (see below.) By combining three libraries into a single pool of phage, three different mutagenesis strategies are tested simultaneously to enhance the probability of finding scFv variants with the desired specificity change.

Error-prone PCR is intended to generate, on average, one amino acid substitution per mutant. For the scFv gene considered here, only about 4500 single-mutants and over 10 million double-mutants are possible. Thus, a library of approximately 107 error-prone mutants can encompass most of the possible single and double-mutants of the scFv gene. Error-prone PCR conditions can be similar to those described in Delagrave et al., Protein Engineering, 2001, 14,261-7, or Leung et al., 1989, supra.

Each of the two combinatorial libraries theoretically encompasses over 4 million different amino acid sequences (21⁵, including stop codons; actual theoretical complexities are somewhat higher due to the degenerate nature of the genetic code.) Again, a library of 10⁷ mutants can be sufficient to efficiently sample each combinatorial population. Such numbers are well within what can be accomplished in most laboratories. To make these libraries, as described in FIG. 1 of Barbas et al., Proc. Natl. Acad. Sci. USA, 1992, 89, 4457, a degenerate oligonucleotide can be used to produce a mutated heavy-chain variable domain PCR product which can then be assembled by fusion PCR with non-mutated light-chain variable domain, and the resulting mutated scFv gene cloned into the phage display vector, much as the original scFv insert. Alternatively, degenerate oligonucleotides can be cloned (combinatorial cassette mutagenesis) as described for instance in Delagrave, et al., Biotechnology (N Y), 1993, 11, 1548 or Oliphant et al., Proc. Natl. Acad. Sci. USA, 1989, 86, 9094. Randomly picked clones from each library can be sequenced according to standard methods to verify that mutations are occurring at the expected rates and positions. Phage can then be rescued from each library and pooled.

Multiwell plates coated with 1 μg/well of MARM antigen (F protein variant) can be used for affinity selection of the pooled scFv variant phage libraries described above. (A possible shortcut is to pan with MARM-infected cell lysate rather than purified F protein. To avoid the amplification of phage binding to irrelevant cellular proteins, phage libraries would be pre-adsorbed to uninfected cell lysate before binding to MARM-infected lysate. However, the preferred route is to use purified F protein.) A suspension of phage can be added to the coated well, the well washed and any remaining phage eluted per standard protocols (e.g., Barbas et al., Phage Display: A Laboratory Manual, Cold Spring Harbor Laboratory Press: Cold Spring Harbor, N.Y., 2001). Eluted phage can be amplified, ready for use in a further round of panning or for individual characterization. This process can be repeated 1 to 5 times. Eluted phage from the last panning round(s) can be tested individually by phage ELISA or grown in a non-amber-suppressor strain of E. coli and their culture supernatants tested by ELISA using anti-E-tag antibody (Amersham Biosciences). Clones showing good binding affinity towards MARM F protein can then be tested for neutralization potency, via neutralization assays described in, for example, Delagrave et al., supra or Anderson, et al., J. Clin. Microbiol., 1985, 22, 1050.

If the scFv variants isolated from the above libraries show less-than-ideal antiviral activity, they can be recombined according to published methods (e.g., via shuffling such as described in Stemmer, Nature, 1994, 370, 389 and Zhao, et al., Nature Biotech., 1998, 16, 258, or through REM such as described in Delagrave, et al., 1993, supra; Arkin, et al., Proc, Natl. Acad. Sci. USA, 1992, 89, 7811; and Delagrave, et al., Prot. Eng., 1993, 6, 327. The resulting recombined library can again be panned to yield further scFv variants. The neutralization potency of these scFv variants can then be tested. Past experience with shuffling has shown that this method is effective at providing new variants showing additional improvements in activity, potentially providing at least one new scFv capable of neutralizing the MARM. The new scFv(s) can be characterized by DNA sequencing according to standard protocols. The sequences of the selected scFv variants will indicate the magnitude and types of changes necessary to ‘catch up’ with escape mutants. Such information could be valuable to biopharmaceutical research, particularly if viral resistance to a monoclonal antibody drug becomes a clinically significant problem in the future.

Coevolution Iterations

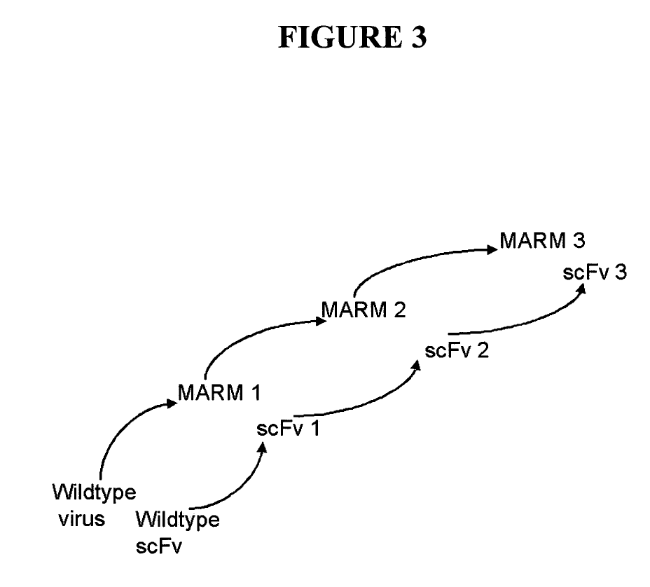

The two-step in vitro coevolution process illustrated in FIG. 1 can be iterated to yield a panel (collection) of MARMs and corresponding scFv variants (FIG. 3). Once a variant scFv, e.g., scFv1, has been found which overcomes the observed resistance of MARM1 to neutralization, scFv1 is then used to isolate a higher-order MARM (MARM2 in FIG. 3). FIG. 3 depicts how the process described, for example in FIG. 1, can be carried out iteratively (multiple cycles). In doing so a panel (collection) of antibodies and a corresponding panel (collection) of resistant virus strains (MARMS) are generated. Any antibody variant can be tested against all viral strains to determine which antibody is most broadly neutralizing.

In order to achieve iteration experimentally, the variant scFv (scFv1 in the above example) can be purified. Purification has been done repeatedly in the past with wildtype and humanized variants (e.g., Guirakhoo, et al., Immunotechnology, 1996, 2, 219 and Delagrave, et al., 1999, supra). The procedure involves culture of recombinant E. coli clones, induction with IPTG, removal of cells, concentration of culture supernatant using ultrafiltration and isolation of scFv via immobilized metal ion chromatography (IMAC) made possible by the poly-histidine tag fused to the scFv carboxy terminus. Milligrams of pure protein can be isolated in this way in a matter of days. The purified scFv is then used to select higher-order MARMs as described above. Once a higher-order MARM is generated (e.g., MARM2), its antigen is used to select a new generation scfv variant (scFv2) capable of neutralizing MARM2. According to the scheme illustrated in FIGS. 1 and 3, the libraries (populations) of scFv variants used to obtain scFv2 can be derived from the DNA of scFv1 using the same procedures employed to generate scFv1.

While not wishing to be bound by theory, it is possible to interpret the results of the process described above in terms of an existing theory of coevolution. Three different outcomes can be predicted for this type of evolutionary race: 1) extinction, wherein one of the coevolving species out-competes the other (in this case, it would be difficult to evolve a MARM resistant to an evolved scFv, or vice versa); 2) equilibrium, in which both species reach a local fitness maximum and can improve no further (neither the scFv nor the MARM could be improved in its neutralization potency or neutralization resistance, respectively); or 3) cyclical, wherein the species oscillate between different mutated states, such as, for instance, MARM1 in FIG. 3 can revert to wildtype when confronted with the scFv which has been evolved to neutralize it. See, e.g., Dawkins, et al., Proc. R. Soc. Lond. Biol. Sci., 1979, 205, 489.

### Example 2

### Suppressing Drug Resistance with Coevolved Antibodies

In view of the coevolution experiments described above, three panels of MARMs and three panels of scFv variants can be available for further studies. By definition, each of the antibodies in the panel is at least capable of neutralizing the MARM used to select it. Therefore, if one were to mix RSV with an antibody and this antibody's coevolved descendents (e.g., scFv1, scFv2 and scFv3 as shown in FIG. 3) and then culture this neutralized virus in vitro, the appearance of MARMs should be suppressed or slowed down. A mixture of coevolved antibodies, if successful at suppressing escape mutant evolution both in vitro and in vivo, could potentially be used as a drug cocktail which would be substantially impervious to the problem or drug resistance.

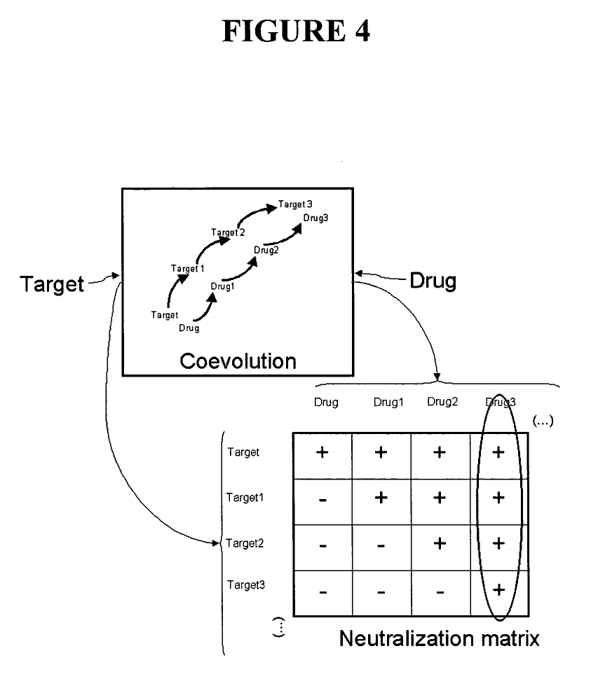

Secondly, coevolved antibodies can also be a source of genetic material that can be manipulated (e.g., via DNA shuffling or recursive ensemble mutagenesis (REM)) to yield new antibody variants having broadened specificity and resilience against the problem of resistance. Cross-reactivity (e.g., determined by cross-testing) of coevolved antibodies (FIG. 4) can be determined and this information can be used to prepare reduced-complexity cocktails (i.e., containing fewer antibodies), that can be tested in vitro for their ability to suppress MARM evolution. Reduced-complexity cocktails can be valuable as drugs because they would provide reduced susceptibility to resistance evolution while decreasing the efforts and resources required to synthesize, purify and dose several scFvs.

FIG. 4 shows the characterization of coevolved antibody specificity. For example, an antibody drug and its target pathogen can be coevolved. The mutants and scFv variants generated in the course of the coevolution experiment can then be tested in a “neutralization matrix.” A “+” indicates significant neutralization potency. The circled column indicates an antibody effective against all strains. This figures shows only one of many possible scenarios.

Assuming, for example, that three iterations of coevolution are carried out with a particular antibody-epitope pair, the neutralization potency of three variants and wildtype scFv can be tested against three MARMs and wildtype RSV (FIG. 3). This procedure can be carried out by performing microneutralization assays (as previously described) in which serial dilutions of antibody (purified and quantitated) are incubated with a constant titer of input virus. The concentrations at which 50% (EC₅₀) and 90% (EC₅₀) of input virus is neutralized are determined for every scFv tested. This quantitative information is then represented in tabular form as schematically illustrated in FIG. 4.

By default (i.e., because of the way the scFv variants were isolated), each scFv can (i) recognize the antigen that was used to select it via phage display, and (ii) neutralize the MARM from which this antigen was isolated (i.e., scFv1 neutralizes MARM1, scFv2 neutralizes MARM2, etc.). It is possible that no single scFv variant will neutralize all viral strains effectively. However, the results of Puntoriero and Rosin indicate that at least some of the members of a diverse population of related molecules can demonstrate cross-reactivity with other strains (Rosin et al., Artif. Life, 1998, 4, 41; Rosin, et al., Proc. Natl. Acad. Sci. USA, 1999, 96, 1369; Rosin et al., J. Mol. Biol., 1999, 287, 77; Puntoriero, et al., Embo J., 1998, 17, 3521. Also, Barbas et al. report that in vitro evolution of anti-HIV Fabs can yield variants with broadened strain cross-reactivity despite the fact that affinity improvement was the only goal of that study, some of the selected variants fortuitously showed increased strain cross-reactivity (Fab 3B3 neutralizes an additional 4 wild isolates in comparison with the parent Fab) in addition to increased affinity and neutralization potency (Barbas et al., Proc. Natl. Acad. Sci. USA, 1994, 91, 3809). Therefore, it is feasible that at least one of the coevolved scFv variants can recognize more than just one MARM. Such a broader-specificity variant can be recombined with the other scFvs, for example using DNA shuffling and REM, to generate further scFv variants that would again be evaluated for the ability to neutralize the broadest possible cross-section of resistant viruses.

### Example 3

### scFv Recombination

This example shows how to create scFv variants with broadened MARM cross-reactivity. Information contained in the ‘neutralization matrix’ (FIG. 4) can be used to select a set of scFv genes which can be recombined. This set can comprise the smallest number of scFvs required to neutralize all MARMs and wildtype RSV. If none of the scFvs have this property, all their genes can be recombined. Recombination can be achieved by (i) synthesizing optimized degenerate oligonucleotides using the methods of REM; and, (ii) using these oligonucleotides as primers to generate scFv PCR products under conditions which favor recombination of sequences according to methods described previously (Zhao et al., 1998, supra). The resulting library of recombined scFv genes can be cloned as described above into a phage display vector. The phage library can be panned twice against wildtype RSV F protein, then twice against F protein isolated from MARM1, MARM2 and MARM3, for a total of 8 rounds of panning. The eluted phage from the last round will be expressed in a non-amber-suppressor strain and randomly selected clones expressing soluble scFv will be tested by ELISA for their ability to recognize the broadest range of coevolved RSV strains. Clones showing broadened strain specificity will then be tested for their ability to neutralize these strains in vitro. The ideal result would be a single scFv capable of neutralizing all MARMs and wildtype, but any variants with increased cross-reactivity may be useful to prepare a reduced-complexity antibody cocktail.

Finally, a reduced complexity cocktail (or a single antibody, if one is found having the desired neutralization profile) can be tested for its ability to suppress MARM evolution. The intended use of such a cocktail is to protect susceptible hosts from infection by wildtype virus and any of the resistant mutants which might arise as a result of treatment with the original antibody. It is expected that the procedures proposed herein will lead to new ways of anticipating drug resistance evolution in a clinical setting, and new ways of providing drugs that are capable of neutralizing resistant strains or even preventing their appearance in treated populations.

### Example 4

### Coevolution of Antibody/MARM

An antibody can be used to select for one or more MARMs (resistant to the antibody). The resulting MARMs can be used to isolate second-generation antibodies (that are effective against the MARMs), derived from the original antibody according to any appropriate method such as given in Example 1 (“Overcoming Resistance”), that can neutralize these MARMs effectively. The original antibody and the second-generation antibodies can be used as a cocktail to prevent or treat infection in humans or as a diagnostic tool.

Alternatively, the original antibody and the second-generation antibodies can be recombined according to methods such as DNA shuffling or REM. The resulting library of antibody variants can be selected for new antibodies having the ability to neutralize more than one of the viruses in the group comprising wildtype virus and the MARMs selected in the above paragraph. The purpose of such new antibodies can be to reduce the complexity of the cocktail described in the previous paragraph. That is, any single antibody capable of doing the job of two or more individual antibodies can be used to replace these two or more antibodies in the cocktail, thereby decreasing the number of antibodies required to carry out the function of the cocktail as a therapeutic, prophylactic or diagnostic tool.

### Example 5

Coevolution of Small Molecule Drug and its Target

Vancomycin (e.g., a neutralizing agent) and vancomycin-resistant Staphylococcus aureus (VRSA, the target, as reported by Chang et al., New Engl. J. Med., 2003, 348, 1342). More specifically, vancomycin's molecular target is the D-Ala-D-Ala moiety of the immature cell wall of bacteria. Vancomycin binding to this molecular target is inhibited by alteration of the cell wall peptide to D-Ala-D-Lac (where D-Lac is D-lactic acid). This change in the cell wall peptide is mediated by the enzyme D-Ala-D-Lac ligase, encoded by the gene VanA, which mediates vancomycin resistance in enterococci and VRSA. Therefore, vancomycin resistance can be overcome by creating derivatives of vancomycin having altered properties (e.g., the ability to bind D-Ala-D-Lac). Moreover, resistance to these new derivatives of vancomycin can also occur via point mutations in VanA. Alternatively, other possibly more important mechanisms causing mutation and thickening of the cell wall in so-called hetero-VRSA lead to VRSA, as described by Hiramatsu, in Lancet Infect. Dis., 2001, 1, 147.

Hetero-VRSA are grown (diversification) in the presence of vancomycin (selection) to identify for clones resistant to the drug. These clones are called VRSA, as defined by Hiramatsu, 2001, supra. Vancomycin is diversified using a combinatorial synthetic procedure described by Nicolaou, et al., Chem. Eur. J., 2001, 7, 3798), yielding a library of related but unique vancomycin derivatives. VRSA are tested for susceptibility to the derivatives of vancomycin and a derivative, call it DV1, is found having superior neutralizing (i.e., antibiotic) activity against VRSA. VRSA are grown in the presence of the compound DV1, and if necessary, passaged repeatedly in the presence of increasing concentrations of DV1 until VRSA mutants, call them DV1RSA, are isolated capable of growing in the presence of DV1. DV1 is diversified according to the same methods used to diversify vancomycin, generating a new library of related but unique DV1 derivatives. These compounds are tested for antibiotic activity against DV1RSA. One compound, called DV2, is found having superior activity against DVIRSA. Thus, DV1 and DV2 are two coevolved antibiotics effective against VRSA and against mutants likely to evolve when VRSA are grown in the presence of DV1, respectively. Vancomycin, DV1 and DV2 can be coadministered to treat MRSA (methicillin-resistant Staphylococcus aureus), thereby decreasing the risk of VRSA being selected by vancomycin therapy.

### Example 6

CML and Gleevec Coevolution

Cancer cells from a patient suffering from the ‘blast crisis’ phase of chronic myeloid leukemia (CML) are cultured in vitro in the presence of STI-571, known commercially as Gleevec. Mutants resistant to Gleevec arise spontaneously and are isolated. Using combinatorial synthesis methods, a library of compounds related to Gleevec is created. Individual compounds from this library are tested for the ability to inhibit the growth of Gleevec-resistant mutant cells. A second generation compound, GleevecG2, is identified which is effective at preventing replication of Gleevec-resistant cells. This entire process is optionally iterated by, first, selecting for second generation mutants resistant to GleevecG2 and, second, isolating new compounds, either from the first combinatorial library or from a new combinatorial library derived from compound GleevecG2, effective against the second generation mutants.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of countering the development of resistance in a parent target to a parent neutralizing agent or countering the development of neutralizing activity in a parent neutralizing agent to a parent target comprising coevolving said parent target and said parent neutralizing agent, wherein said coevolving comprises diversifying each of said parent target and said parent neutralizing agent in vitro.

2. The method of claim 1 wherein said diversifying comprises generating a population of targets and neutralizing agents by mutagenesis, recombinant methods, or combinatorial synthetic methods.

3. The method of claim 1 wherein said coevolving comprises diversifying said parent target and said parent neutralizing agent, selecting one or more next generation neutralizing agents and next generation targets from diversified populations resulting from said diversifying, wherein the selected one or more neutralizing agents and targets have improved neutralizing activity and resistance, respectively, and optionally repeating said diversifying and selecting using said one or more next generation neutralizing agents or next generation targets.

4. The method of claim 3 wherein said improved neutralizing activity is neutralizing activity against said parent target or a previously selected next generation target.

5. The method of claim 3 wherein said improved resistance is resistance to a said parent neutralizing agent or a previously selected next generation neutralizing agent.

6. The method of claim 3 wherein said repeating is continued until a neutralizing agent having a desired neutralization profile is identified, until a target having a desired resistance profile is identified, or until no further neutralizing agent or targets having improved neutralizing activity or resistance are identified.

7. The method of claim 6 wherein said desired neutralization profile is broad neutralizing activity.

8. The method of claim 6 wherein said desired resistance profile is broad resistance.

9. The method of claim 1 wherein said parent neutralizing agent is a cell, protein, nucleic acid, small molecule, bacterium, or virus.

10. The method of claim 1 wherein said parent neutralizing agent is an antibody.

11. The method of claim 1 wherein said parent target is a cell, protein, nucleic acid, small molecule, bacterium, or virus.

12. The method of claim 1 wherein said parent target is an antigen.

13. A method of preparing a neutralizing agent having a desired neutralization profile or a target having a desired resistance profile comprising:
a) coevolving a parent target and a parent neutralizing agent pair to generate a collection of evolved neutralizing agents and a collection of evolved targets;
b) cross testing members of said collections for neutralizing activity or resistance; and
c) identifying at least one member of said collection of evolved neutralizing agents having said desired neutralizing profile or identifying at least one member of said collection of evolved targets having said desired resistance profile.

14. The method of claim 13 further comprising optionally repeating said coevolving using the identified members of step c).

15. The method of claim 13 wherein said desired neutralizing profile is broad neutralizing activity.

16. The method of claim 13 wherein said at least one neutralizing agent identified in step c) has neutralizing activity against the greatest number of tested members of said collection of evolved targets.

17. The method of claim 13 wherein said desired resistance profile is broad resistance.

18. The method of claim 17 wherein said broad resistance is resistance to the greatest number of tested members of said collection of evolved neutralizing agents.

19. The method of claim 13 wherein said parent neutralizing agent or parent target is a cell, protein, nucleic acid, small molecule, bacterium, or virus.

20. The method of claim 13 wherein said parent neutralizing agent is an antibody.

21. The method of claim 13 wherein said parent target is an antigen, cell, bacterium, or virus.

22. The method of claim 13 wherein said parent target is an antigen.

23. The method of claim 13 further comprising repeating steps a) to c) using the identified neutralizing agents or targets of step c) in said coevolving.

24. The method of claim 13 wherein said coevolving comprises diversifying said parent target and said parent neutralizing agent in vitro, selecting one or more next generation neutralizing agents and targets from populations resulting from said diversifying, wherein the selected one or more neutralizing agents and targets have new or improved neutralizing activity or resistance, respectively, and optionally repeating said diversifying and selecting using said one or more selected next generation neutralizing agents and next generation targets, wherein said parent neutralizing agent and the selected one or more neutralizing agents form a collection of evolved neutralizing agents and said parent target and the one or more selected targets form a collection of evolved targets.

25. The method of claim 13 wherein said coevolving comprises:
i) contacting said parent neutralizing agent with members of an initial population of targets generated by diversifying said parent target in vitro;
ii) selecting one or more resistant targets within said initial population, wherein said one or more resistant targets has new or improved resistance to said parent neutralizing agent;
iii) diversifying said parent neutralizing agent to create a population of neutralizing agents;
iv) contacting members of said population of neutralizing agents with said one or more resistant targets selected in step ii);
v) selecting one or more further neutralizing agents from said population of neutralizing agents, wherein said one or more further neutralizing agents has new or improved neutralizing activity against said one or more resistant targets;
vi) optionally repeating steps i) and ii) or optionally repeating steps i), ii), iii), iv), and v) using said one or more further neutralizing agents in place of said neutralizing agent and using said one or more resistant targets in place of said target,
wherein said parent neutralizing agent and said one or more further neutralizing agents are members of a collection of evolved neutralizing agents, and wherein said parent target and said one or more resistant targets are members of a collection of evolved targets.

26. A method of coevolving, comprising:
a) diversifying in vitro a parent target and selecting at least one next generation target from a diversified population of targets resulting from said diversifying, wherein the selected at least one target has new or improved resistance to a parent neutralizing agent having neutralizing activity against said parent target; and
b) diversifying in vitro said parent neutralizing agent and selecting at least one next generation neutralizing agent from a diversified population of neutralizing agents resulting from said diversifying, wherein the selected at least one neutralizing agent has new or improved neutralizing activity against said selected at least one target.

27. The method of claim 26 further comprising repeating step a) or repeating steps a) and b) one or more times, wherein each diversifying step produces a diversified population of the at least one target or neutralizing agent that was previously selected.

28. A method of coevolving, comprising:
a) diversifying a parent neutralizing agent and selecting at least one next generation neutralizing agent from a diversified population of neutralizing agents resulting from said diversifying, wherein the selected at least one neutralizing agent has new or improved neutralizing activity against a parent target having resistance to said parent neutralizing agent; and
b) diversifying said parent target and selecting at least one next generation target from a diversified population of targets resulting from said diversifying, wherein the selected at least one target has new or improved resistance to said selected at least one neutralizing agent.

29. The method of claim 28 further comprising repeating step a) or repeating steps a) and b) one or more times, wherein each diversifying step produces a diversified population of the at least one target or neutralizing agent that was previously selected.

30. A method of coevolving a parent target and parent neutralizing agent to produce a collection of evolved targets and a collection of evolved neutralizing agents, comprising:
i) contacting said parent neutralizing agent with members of an initial population of targets generated from said parent target;
ii) selecting one or more resistant targets within said initial population, wherein said one or more resistant targets has new or improved resistance to said parent neutralizing agent;
iii) diversifying said parent neutralizing agent to create a population of neutralizing agents;
iv) contacting members of said population of neutralizing agents with said one or more resistant targets selected in step ii);
v) selecting one or more further neutralizing agents from said population of neutralizing agents, wherein said one or more further neutralizing agents have new or improved neutralizing activity against said one or more resistant targets;
vi) optionally repeating steps i) and ii) or optionally repeating steps i), ii), iii), iv), and v) using said one or more further neutralizing agents in place of said parent neutralizing agent and using said one or more resistant targets in place of said parent target,
wherein said parent neutralizing agent and said further neutralizing agents are members of a collection of evolved neutralizing agents, and wherein said parent target and said resistant targets are members of a collection of evolved targets.

31. A method of treating or preventing a disease associated with a parent target, comprising administering to a patient having or predisposed to said disease a therapeutically effective amount of a neutralizing agent prepared by the method of claim 30, wherein said neutralizing agent is a small molecule.

32. The method of claim 31 wherein said disease is a bacterial or viral infection.

33. The method of claim 31 wherein said disease is cancer.

34. A method of developing a desired characteristic in a parent antibody or parent target comprising coevolving said parent antibody and parent target, wherein said coevolving comprises diversifying each of said parent antibody or parent target in vitro.

35. The method of claim 34 wherein said diversifying is carried out by mutagenesis or recombinant methods.

36. A method of preparing an antibody having broadened neutralizing activity compared with a parent antibody comprising coevolving said parent antibody and a parent target.

37. The method of claim 36 wherein said parent target is a pathogen or protein.

38. An antibody prepared by the method of claim 36.

39. A composition comprising an antibody and a pharmaceutically acceptable carrier, wherein said antibody is prepared by the method of claim 36.

40. A method of treating or preventing a disease associated with a parent target, comprising administering to a patient having or predisposed to said disease a therapeutically effective amount of antibody prepared by the method of claim 36.

41. The method of claim 40 wherein said disease is a bacterial or viral infection.

42. The method of claim 40 wherein said disease is cancer.

43. A method of preparing an antigen having broadened antigenic activity compared with a parent antigen comprising coevolving said parent antigen and a parent neutralizing agent.

44. An antigen prepared by the method of claim 43.

45. A composition comprising an antigen and a pharmaceutically acceptable carrier, wherein said antigen is prepared by the method of claim 43.

46. A method of preventing a disease or condition associated with a parent antigen, comprising administering to a patient having or predisposed to said disease or condition an amount of preventative antigen sufficient to prevent said disease or condition, wherein said preventative antigen is prepared by the method of claim 43.

47. The method of claim 46 wherein said preventative antigen has broadened antigenic activity compared with said parent antigen.

48. The method of claim 46 wherein said disease or condition is a bacterial or viral infection.

49. The method of claim 46 wherein said disease or condition is cancer.

50. The method of claim 46 wherein said disease or condition is an allergy.
